(19) United States
(12) Patent Application Publication
(10) Pub. No.: US 2014/0025044 A1
(43) Pub. Date: Jan. 23, 2014

(54) TORQUEABLE CATHETER HUB AND RELATED METHODS OF USE

(71) Applicant: Boston Scientific Scimed, Inc., Maple Grove, MN (US)

(72) Inventors: NATHAN ZAMARRIPA, SUDBURY, MA (US); JAY R. PATEL, ACTON, MA (US); GARY B. TRENDEL, HOPKINTON, MA (US); CHRISTOPHER L. NARDONE, SOUTH GRAFTON, MA (US)

(21) Appl. No.: 13/946,752

(22) Filed: Jul. 19, 2013

Related U.S. Application Data
(60) Provisional application No. 61/673,679, filed on Jul. 19, 2012.

(51) Int. Cl. A61M 25/00

(52) U.S. Cl. CPC A61M 25/0097
USPC 604/533

(57) ABSTRACT

Medical devices, catheter hubs, and methods for making and using the same are disclosed. An example medical device may include a catheter shaft having a proximal end, a distal end, and a lumen extending between the proximal end and the distal end. A hub may be coupled to the proximal end of the catheter shaft. The hub may include a hub body and a rotatable tip member that rotatable with respect to the hub body. A distal end of the rotatable tip member may be coupled to the proximal end of the catheter shaft. The rotatable tip member may be configured to transmit torque applied to the rotatable tip member to the catheter shaft.

### CROSS-REFERENCE TO RELATED APPLICATIONS

This application claims priority under 35 U.S.C. §119 to U.S. Provisional Application Ser. No. 61/673,679, filed Jul. 19, 2012, the entirety of which is incorporated herein by reference.

### TECHNICAL FIELD

The present disclosure relates generally to medical devices and procedures. More particularly, the present disclosure pertains to torquable catheter hubs and medical devices with torquable hubs.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several designs, materials, systems and methods of medical devices, catheter hubs, and medical device and/or catheter assemblies.

An example medical device may include a catheter shaft having a proximal end, a distal end, and a lumen extending between the proximal end and the distal end. A hub may be coupled to the proximal end of the catheter shaft. The hub may include a hub body and a rotatable tip member that rotatable with respect to the hub body. A distal end of the rotatable tip member may be coupled to the proximal end of the catheter shaft. The rotatable tip member may be configured to transmit torque applied to the rotatable tip member to the catheter shaft.

An example catheter assembly may include a hub body having a proximal end and a distal end. The hub body may include a plurality of fins extending at least partially between the proximal end and distal end. A catheter shaft may be attached to the distal end of the hub body. The fins may be configured to transfer torque from the hub body to the catheter shaft.

An example method for rotating a catheter shaft relative to a hub is also disclosed. The method may include providing a catheter assembly including a hub attached to the catheter shaft. The hub may have a hub body including a proximal end, a distal end, and a lumen extending therebetween. The hub may also include a rotatable tip member. The rotatable tip member may be rotatable relative to the hub body. The method may also include advancing the catheter shaft through a body lumen to a position adjacent to an area of interest and rotating the rotatable tip member. Rotating the rotatable tip member may transfer torque from the rotatable tip member to the catheter shaft and may rotate the catheter shaft.

The above summary of some embodiments is not intended to describe each disclosed embodiment or every implementation of the present disclosure. The Figures, and Detailed Description, which follow, more particularly exemplify the embodiments of the present disclosure.

### BRIEF DESCRIPTION OF THE DRAWINGS

The accompanying drawings, which are incorporated in and constitute a part of this specification, illustrate exemplary embodiments of the present disclosure and together with the detailed description, serve to explain the principles of the disclosure.

FIG. 1 is a perspective view of an exemplary catheter hub of the present disclosure.

FIG. 2 is a cross-sectional view of the catheter hub of FIG. 1.

FIG. 3A is an exploded schematic view of a catheter assembly including a hub, a strain relief and a catheter shaft of the present disclosure, and FIG. 3B shows a detailed view of the catheter shaft having slots.

FIG. 4 illustrates another example catheter hub.

FIG. 5 illustrates another example catheter hub.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5 etc.).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosure.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

Embodiments of the present disclosure may include a medical device, hubs for medical devices, and medical device assemblies. The medical device may take the form of a catheter having a hub disposed at its proximal end, while the catheter distal end may remain inside the body of the patient. The hub may assist in positioning the catheter proximate to a target position, such as a lesion within a blood vessel or similar bodily cavity. For adequate and appropriate positioning of the catheter, the present disclosure discloses a torqueable hub, having a number of fins that extend along the hub surface. The fins may be configured to facilitate the transmission of torque from the hub to the catheter shaft.

Many of the following examples illustrate implementations in which the catheter may be employed to navigate blood vessels. It will be understood that this choice is merely exemplary and the catheter shaft may be used in any desired body location requiring diagnostic or therapeutic modalities without departing from the scope of the present disclosure.

For purposes of this disclosure, “proximal” refers to the end closer to the device operator during use, and “distal” refers to the end further from the device operator during use.

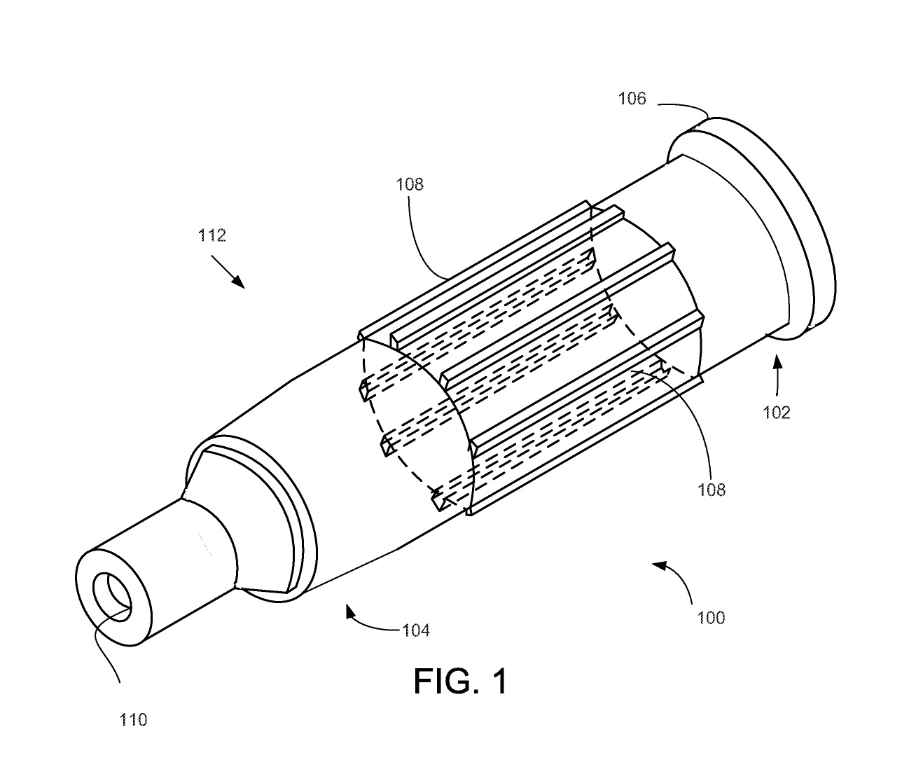

FIG. 1 is a perspective view of an illustrative catheter hub 100, according to an embodiment of the disclosure. As shown the hub 100 may include a proximal end 102, a distal end 104, and an integral connector 106 (or port). One or more fins 108 (e.g., one, two, three, four, five, six, seven, eight, nine, ten, or more) may be disposed on the hub 100. In general, the fins 108 may help transmit torque applied to the catheter hub 100 to devices attached to the catheter hub 100 (e.g., a catheter or catheter shaft).

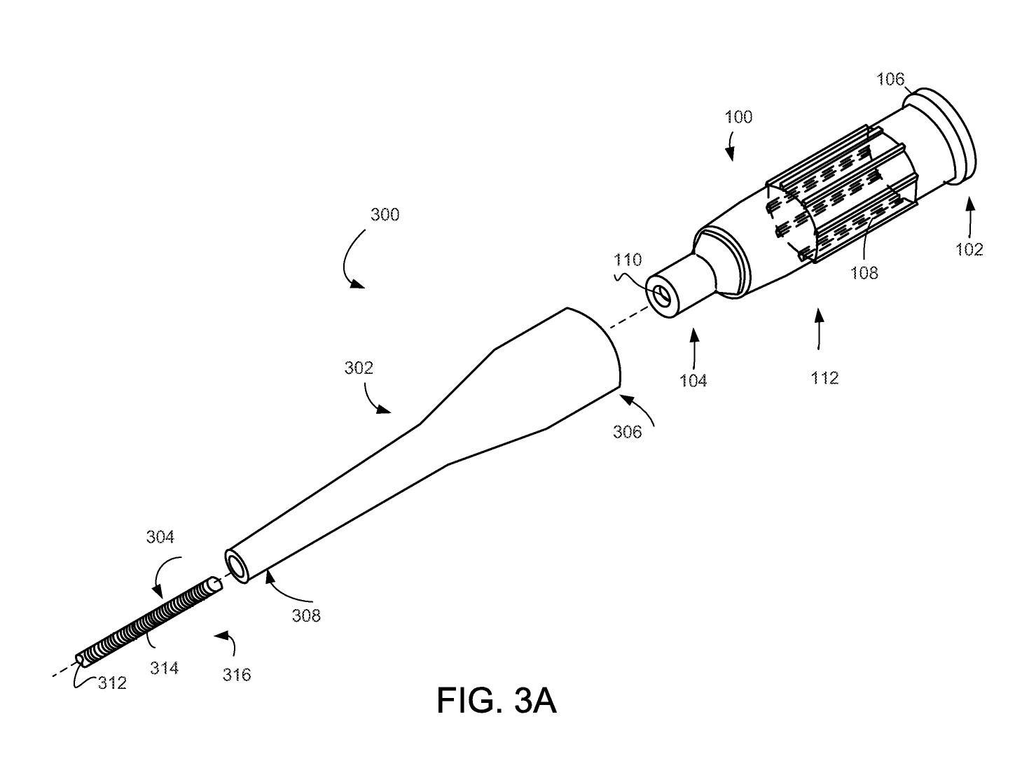

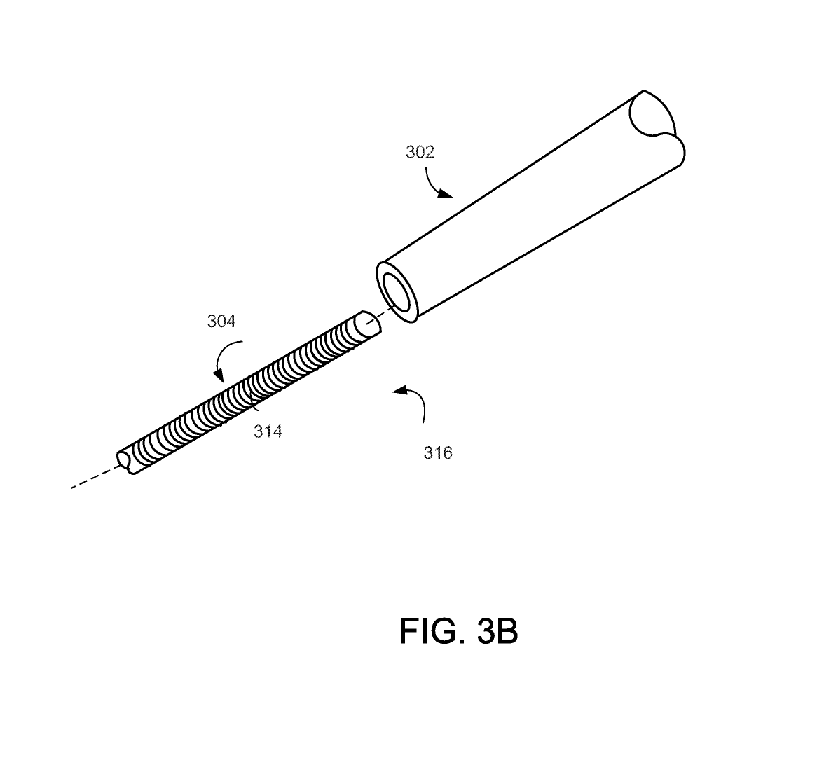

At the proximal end 102, the integral connector 106 may be disposed for attachment to various medical accessories, such as infusion devices, or the like to the hub 100. The integral connector 106 may be a touhy-borst connector, a threaded connector, a mechanical connector, or the like and it may include extending flanges, bayonets, or other fitting mechanisms. These or other fittings may be employed as desired and appropriate components may be chosen to suit a particular design and/or intervention. The catheter shaft or tube (see, for example, catheter shaft 304 in FIGS. 3A-3B) may be attached at the distal end 104 of the hub 100.

As indicated above, the hub 100 may include the fins 108, formed on the body 112 of the hub 100, which may help transmit torque from an attached catheter shaft, as will be discussed in detail below. The fins 108 may extend at least partially between the proximal end 102 to the distal end 104. The fins 108 may be configured so that torque applied to the fins 108 is transmitted to the attached catheter shaft, which then provides the physician more control and steerability of the catheter shaft (including steering of the distal tip of the catheter shaft). Any suitable number of fins 108 may be utilized with the hub 100. In one configuration, the hub 100 may include eight fins 108 (as shown) for providing appropriate rotation of the catheter shaft. This is not intended to be limiting as a number of different hubs are contemplated that utilize a variety of different numbers of fins 108. The fins 108 represented with solid line may be formed on the upper part of the hub 100, while the fins 108 shown with dashed line may be fixed to its lower part. The fins 108 formed on the hub body 112 may vary in shape, size, and/or configuration. For example, in at least some embodiments, the fins 108 may have a straight or curved shaped. Along with the above features, the fins 108 may be cylindrical, circular or any other shape appropriate for use in the intended environment. In some aspects, the hub 100 may include other steering or torqueing mechanisms for positioning the distal tip of the catheter shaft (see, for example, FIGS. 3A-3B). Additionally, the hub 100 may include a lumen 110, which will be discussed in detail below.

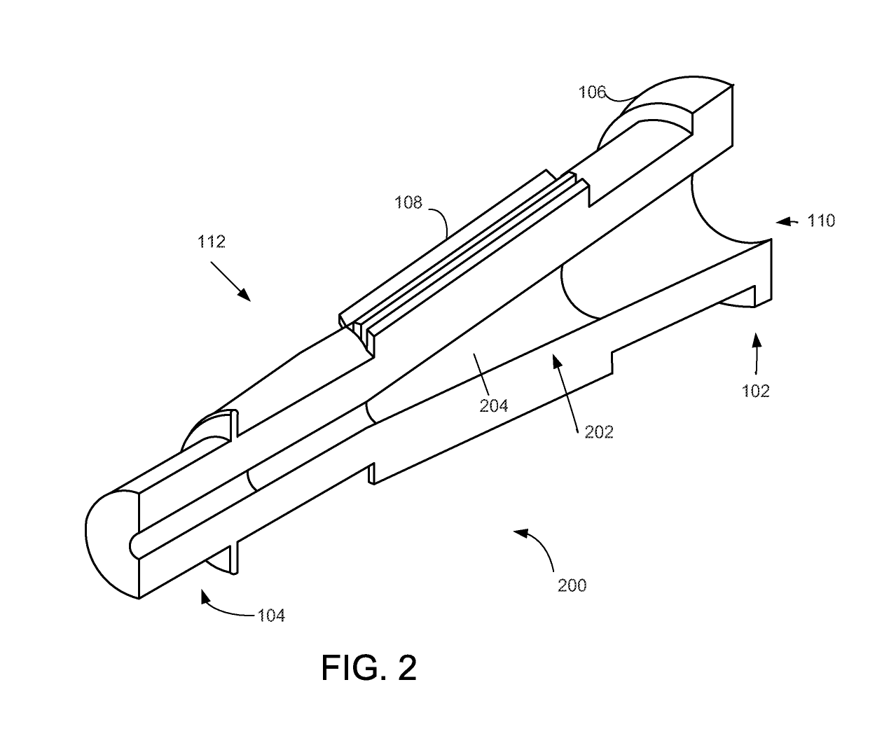

FIG. 2 is a cross-sectional view 200 of the hub discussed above, according to an embodiment of the disclosure. The components of the hub 100 including the proximal end 102, distal end 104, integral connector 106, and fins 108 have been discussed above. In particular, FIG. 2 shows a cross-section of the lumen 110, which may extend longitudinally from the proximal end 102 to the distal end 104 and may be in fluid communication with a lumen of the catheter shaft (see, for example, FIGS. 3A-3B). As shown, the lumen 110 may be defined by the inner wall 202 of the hub 100. Further, the lumen 110 may have a tapered shaped channel 204 that allows the appropriate injected material flows from the hub 100 to the catheter lumen (see, for example, FIGS. 3A-3B) and consequently flows inside the patient's body (not shown). In some embodiments, the channel 204 may provide a passage for therapeutic or surgical devices including guide wires. The channel 204 may guide the inserted device into the lumen of the catheter shaft so that the device can be advanced through the catheter shaft (see, for example, FIGS. 3A-3B).

As depicted, the fins 108 may be disposed on the outer surface/body 112 of the hub 100. Each fin may run longitudinally, and each may run, for example, nearly the entire length of the catheter hub 100. These are just examples. Other configurations are contemplated. The set of fins 108 may be arranged around the circumference of the hub 100, either completely or partially. The fins 108 extend outward a distance from the body 112 of the hub 100, adapted for convenient application of torque by a user. The rotational forces applied to the fins 108 may translate to the catheter shaft.

FIG. 3A shows a catheter assembly 300 having hub 100, a strain relief 302, and a catheter shaft 304. As discussed above, the hub 100 may include the proximal or distal ends as 102, 104 respectively, fins 108, lumen 110 and connector/port 106. In some embodiments, the hub 100 may include multiple connections or ports. The proximal end 102 may include the port 106 for connection to various medical accessories, while the distal end 104 may be coupled to the catheter shaft 304 (discussed below in detail). Moreover, the hub body 112 may include the fins 108 for transmitting torque to the catheter shaft 304. The term fins 108 is intended to include any of a wide variety of structures which are capable of transmitting a rotational torque throughout the length of a catheter shaft 304. Also, the fins 108 may be used as a gripping surface. In some instances, the hub 100 may employ a strain relief 302.

Strain relief 302 may extend between and be connected to hub 100 and catheter shaft 304, at proximal and distal ends 306, 308, respectively, of the strain relief 302. In one embodiment, the strain relief 302 may be mechanically attached to the hub 100 or through any other suitable method such as an adhesive bond, thermal bond, and the like. As shown, the strain relief 302 may be tapered from its proximal end 306 to the distal end 308. This may allow the strain relief 302 to help form gentle transition flexibility adjacent to the attachment point of the catheter shaft 304, which may help to relieve strain or other forces at this connection point.

The catheter shaft 304 may be connected to the distal end 104 of the hub 100. Typically, the strain relief 302 is disposed over the catheter shaft 304 (e.g., including the connection point) to relieve strain (e.g., kinking) at the connection point. The catheter shaft 304 may be a generally long, flexible tube that may be inserted into the body for a medical diagnosis and/or treatment, for example. As shown, the catheter shaft 304 may include a proximal end 316 and a distal end (not shown). The distal end of the catheter shaft 304 may be softer or more flexible than the proximal end 316 so the catheter shaft 304 may more easily navigate inside the patient's body.

As shown in FIG. 3B, the catheter shaft 304 may have a plurality of slots 314 formed therein. The slots 314 cut into a tubular shaft providing for greater flexibility in the shaft, while still allowing for torque transmission along the shaft 304. Various embodiments of arrangements and configurations of slots 314 are contemplated as disclosed herein.

Rotation of hub 100 may transmit the desired torque to the catheter shaft 304. Catheter shaft 304 may be hollow, with a cross-sectional configuration adapted to be received in a desired body lumen, for example, blood vessels or other appropriate body lumens. The form of catheter shaft 304 may vary. For example, the length, diameter, number of lumens, and additional structural features may vary. In some embodiments, the catheter shaft 304 may include a lumen 312 that is in fluid communication with the hub lumen 110. In addition, the catheter shaft 304 may include a balloon or inflatable structure. These are just examples. A variety of forms and/or configurations are contemplated for the catheter shaft 304.

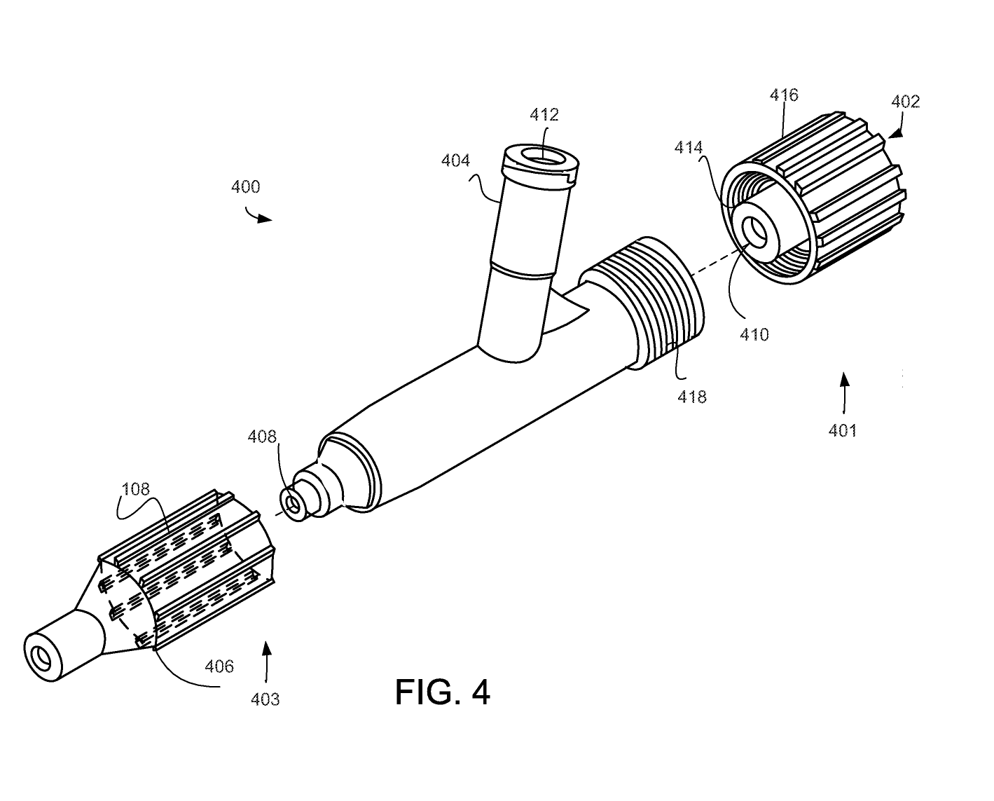

FIG. 4 shows another example hub 400 having a hemostasis valve 402, Y-connector 404, and a rotatable distal tip 406. More particularly, the hub 400 embodiment may show multiple connections at the proximal end in the form of the hemostasis valve 402 and the Y-connector 404. In the illustrated embodiment, hub 400 includes the Y-connector 404, which provides two ports. Hemostasis valve 402 exemplifies one type of connection that may be utilized at one of the ports. If Y-connector 404 is connected to multiple structures, however, its rotation may lead to complications, such as twisting and/or tangling the connected devices, as well as possible structural interference. Such problems can be avoided by employing rotatable distal tip 406 that device rotates independently of the hub body 112, allowing torque to be applied to the catheter shaft 304 while holding the Y-connector 404 essentially stationary.

The hemostasis valve 402 may include a lumen 410 extending from the proximal end to the distal end for introducing the catheter shaft 304 or other medical devices therein. In some embodiments, a guide wire may be inserted through the hemostasis valve 402. The hemostasis valve 402 may further include threads 414 for connection at the proximal end 401. Also, a portion of the proximal end 401 may include threaded connection shown as 418 for appropriate fitting of the hemostasis valve 402. The hemostasis valve 402 may be rotatable for coupling other devices at the proximal end 401. As desired, the hemostasis valve 402 may be attached or detached to/from the proximal end 401. Additionally, the hemostasis valve 402 may be defined as having a circular cross-section. Other suitable cross-sections, however, such as oval, cylindrical, or irregular, may be contemplated. In some embodiments, the hemostasis valve 402 may also include a number fins as 416 for its appropriate rotation.

The hemostasis valve 402 may be coupled to the Y-connector 404 internally, or those two components may be separated by a predetermined distance. Further, the Y-connector 404 may include a lumen 412 extending completely through the Y-connector 404 for therapeutic or surgical purposes, and that lumen may be in fluid communication with the valve lumen 410. A tube may be inserted through connector lumen 412, and the tube may be oriented in the desired direction by rotating or moving the connector 404. In some embodiments, the connector 404 may be helpful in orienting the catheter shaft 304 in a particular direction. Moreover, the Y-connector 404 may be integrally connected to the hub 400 using various fitting mechanism as known in the art. As mentioned above, two devices/structures may be connected to the Y-connector 404. In conventional designs, torquing the catheter shaft 304 torques the entire Y-connector 404, which can lead to complications. Here, catheter shaft 304 may be attached to the rotatable distal tip 406, which in turn attaches the rotatable tip 406, which is coupled to the body of the hub 400. The tip 406 can freely rotate relative to the hub body 112 that structure allows the user to apply torque to the catheter shaft 304 without affecting the hub body. More details of the distal tip 406 are outlined below.

In the illustrated embodiment, the distal tip 406 of the hub 400 may include a number of fins 108 for rotating the distal end (See FIG.3) of the catheter shaft 304 inside the body of patient. More specifically, the distal tip 406 may be rotated with respect to the hub body 112 to guide the attached catheter shaft 304. The distal tip 406 may be operatively coupled to the catheter shaft 304 such that rotating the distal tip 406 may generate torque that is transmitted to the attached catheter shaft 304. As shown, the distal tip 406 may be snap fitted to O-ring locator 408 disposed on the distal end of the hub 400. Additionally, the distal tip 406 may have circular cross-section and may be tapered towards its end portion. In the illustrated embodiment, only the distal tip 406 connected at the distal end 403, may be rotated to guide the catheter shaft 304 (see FIG. 3) to a target site, for example, blood vessels, while the proximal end 401 of the hub 400 may remain stationary. Thus, the illustrated embodiment may provide relative rotation between the body of the hub 400 and the catheter shaft 304 (see, for example, FIGS. 3A-3B).

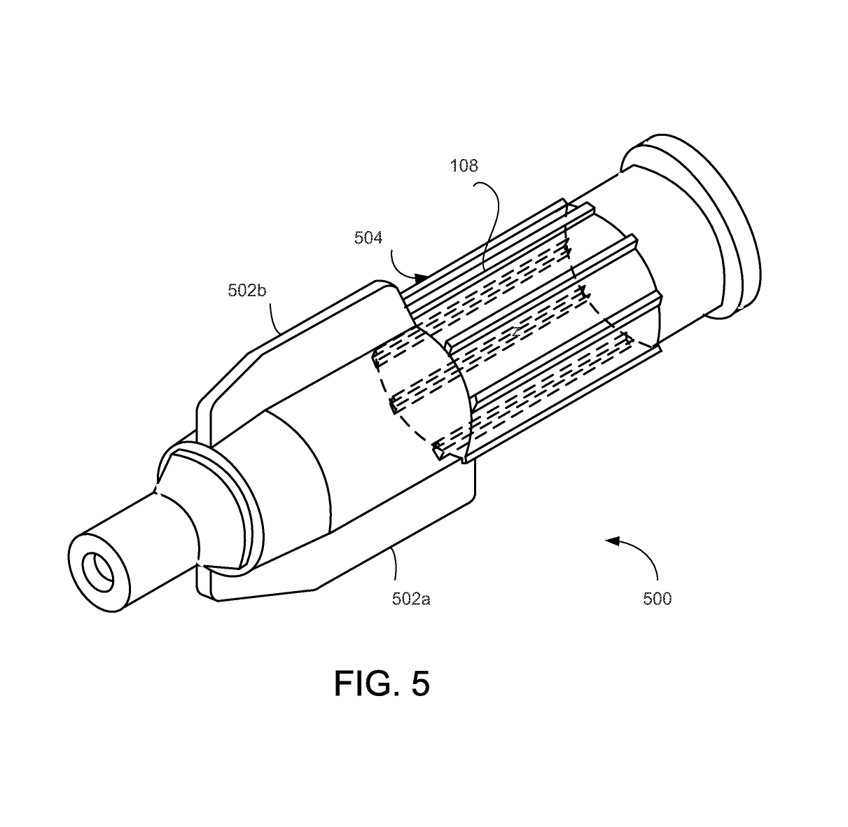

FIG. 5 shows another exemplary hub structure 500, which may be similar in form and function to other hubs disclosed herein. In the illustrated embodiment, the hub 500 may include a pair of extending wings 502a and 502b on its outer surface 504 disposed in opposite directions for forming a gripping area. As shown, the wings 502a/502b may be disposed near to the distal end of the hub 500. However, other positions are also contemplated. In various embodiments, the wings 502a/502b may be formed anywhere on the outer surface 504 of the hub 500. The hub 500 may also have a plurality fins 108 disposed on the outer surface 504 of the hub 500. Further, the hub 500 may be a molded structure, where the wings 502a/502b and the fins 108 are integral with the hub 500. In this example, the fins 108 are disposed proximal of the wings 502a/502b. Other locations are contemplated. The wings 502a/502b and/or fins 108 may be used for turning or moving the hub 500 in a desired direction. In particular, the wings 502a/502b and/or fins 108 may be useful for applying torque to the hub 500, which can then be efficiently transmitted to and along the catheter shaft. The increased surface area of the wings 502a/502b may provide the surgeon with a better grip and thus, provide greater torque for accurately positioning the catheter shaft as discussed above. Moreover, the wings 502a/502b may be flat along its entire length or may be tapered towards its middle portion or substantially circular towards its end portion. The number of wings as shown in the current embodiment is merely illustrative and the number may vary based on the configuration and the intended environment of use.

The materials that can be used for the various components of the hub 100 and/or the catheter assembly 300 may include those commonly associated with medical devices. For example, the catheter assembly 300 including the hub 100, strain relief 302, and the catheter shaft 304 may be made of or otherwise includes metals, polymers, metal-polymer composites, and the like. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. These are just examples.

As indicated above, the arrangement and/or configuration of slots 314 may vary. For example, in some embodiments, at least some, if not all of slots 314 are disposed at the same or a similar angle with respect to the longitudinal axis of catheter shaft 304. As shown, slots 314 can be disposed at an angle that is perpendicular, or substantially perpendicular, and/or can be characterized as being disposed in a plane that is normal to the longitudinal axis of the catheter shaft 304. However, in other embodiments, the slots 314 can be disposed at an angle that is not perpendicular, and/or can be characterized as being disposed in a plane that is not normal to the longitudinal axis of catheter shaft 304. Additionally, a group of one or more slots 314 may be disposed at different angles relative to another group of one or more slots 314. The distribution and/or configuration of the slots 314 can also include, to the extent applicable, any of those disclosed in U.S. Pat. Publication No. US 2004/0181174, the entire disclosure of which is herein incorporated by reference.

The slots 314 may be provided to enhance the flexibility of catheter shaft 304 while still allowing for suitable torque transmission characteristics. The slots 314 may be formed such that one or more rings and/or tube segments interconnected by one or more segments and/or beams that are formed in catheter shaft 304, and such tube segments and beams may include portions of catheter shaft 304 that remain after slots 314 are formed in the body of catheter shaft 304. Such an interconnected structure may act to maintain a relatively high degree of torsional stiffness, while maintaining a desired level of lateral flexibility. In some embodiments, some adjacent slots 314 can be formed such that they include portions that overlap with each other about the circumference of catheter shaft 304. In other embodiments, some adjacent slots 314 can be disposed such that they do not necessarily overlap with each other, but are disposed in a pattern that provides the desired degree of lateral flexibility.

Additionally, the slots 314 can be arranged along the length of, or about the circumference of, the catheter shaft 304 to achieve desired properties. For example, adjacent slots 314, or groups of slots 314, can be arranged in a symmetrical pattern, such as being disposed essentially equally on opposite sides about the circumference of the catheter shaft 304, or can be rotated by an angle relative to each other about the axis of catheter shaft 304. Additionally, adjacent slots 314, or groups of slots 314, may be equally spaced along the length of catheter shaft 304, or can be arranged in an increasing or decreasing density pattern, or can be arranged in a non-symmetric or irregular pattern. Other characteristics, such as slot size, slot shape, and/or slot angle with respect to the longitudinal axis of catheter shaft 304, can also be varied along the length of catheter shaft 304 in order to vary the flexibility or other properties. In other embodiments, moreover, it is contemplated that the portions of the catheter shaft 304, such as a proximal section, or a distal section, or the entire catheter shaft 304, may not include any such slots 314.

As suggested herein, the slots 314 may be formed in groups of two, three, four, five, or more slots 314, which may be located at substantially the same location along the axis of catheter shaft 304. Alternatively, a single slot 314 may be disposed at some or all of these locations. Within the groups of slots 314, there may be included slots 314 that are equal in size (i.e., span the same circumferential distance around catheter shaft 304). In some of these as well as other embodiments, at least some slots 314 in a group are unequal in size (i.e., span a different circumferential distance around catheter shaft 304). Longitudinally adjacent groups of slots 314 may have the same or different configurations. For example, some embodiments of catheter shaft 304 include slots 314 that are equal in size in a first group and then unequally sized in an adjacent group. It can be appreciated that in groups that have two slots 314 that are equal in size and are symmetrically disposed around the tube circumference, the centroid of the pair of beams (i.e., the portion of catheter shaft 304 remaining after slots 314 are formed therein) is coincident with the central axis of catheter shaft 304. Conversely, in groups that have two slots 314 that are unequal in size and whose centroids are directly opposed on the tube circumference, the centroid of the pair of beams can be offset from the central axis of catheter shaft 304. Some embodiments of catheter shaft 304 include only slot groups with centroids that are coincident with the central axis of the catheter shaft 304, only slot groups with centroids that are offset from the central axis of catheter shaft 304, or slot groups with centroids that are coincident with the central axis of catheter shaft 304 in a first group and offset from the central axis of catheter shaft 304 in another group. The amount of offset may vary depending on the depth (or length) of slots 314 and can include other suitable distances.

The slots 314 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the catheter shaft 304 is formed by cutting and/or removing portions of the tube to form slots 314. Some example embodiments of appropriate micromachining methods and other cutting methods, and structures for tubular members and/or catheter shafts including slots 314 and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guide wire may include forming slots 314 in catheter shaft 304 using these or other manufacturing steps.

In at least some embodiments, the slots 314 may be formed in catheter shaft 304 using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow catheter shaft 304 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form catheter shaft 304 without being limited by a minimum cutting blade size. Consequently, catheter shaft 304 may be fabricated for use in neurological devices or other devices where a relatively small size may be desired.

Although the embodiments described above use catheters inserted into blood vessels, those of skill in the art will understand that the principles set out there can be applied to any catheter or endoscopic device where it is deemed advantageous to transmit torque, for example, to the tip of the device. Conversely, constructional details, including manufacturing techniques and materials, are well within the understanding of those of skill in the art and have not been set out in any detail here. These and other modifications and variations may well within the scope of the present disclosure can be envisioned and implemented by those of skill in the art.

Other embodiments of the present disclosure will be apparent to those skilled in the art from consideration of the specification and practice of the embodiments disclosed herein. It is intended that the specification and examples be considered as exemplary only, and departure in form and detail may be made without departing from the scope and spirit of the present disclosure as described in the following claims.

What is claimed is:

1. A medical device comprising:
a catheter shaft having a proximal end, a distal end, and a lumen extending between the proximal end and the distal end;
a hub coupled to the proximal end of the catheter shaft, wherein the hub includes a hub body and a rotatable tip member that rotatable with respect to the hub body;
wherein a distal end of the rotatable tip member is coupled to the proximal end of the catheter shaft; and
wherein the rotatable tip member is configured to transmit torque applied to the rotatable tip member to the catheter shaft.

2. The medical device of claim 1, wherein the proximal end of the hub body includes one or more connectors.

3. The medical device of claim 1, wherein the hub body has a lumen formed therein.

4. The medical device of claim 3, wherein interior of the lumen is tapered.

5. The medical device of claim 1, wherein the hub body includes an O-ring connector at the distal end for securing the rotatable tip member to the hub body.

6. The medical device of claim 1, further comprising one or more fins disposed on the hub body.

7. The medical device of claim 6, wherein the hub body includes eight or more fins.

8. The medical device of claim 1, further comprising one or more wings disposed on the hub body.

9. The medical device of claim 1, further comprising one or more fins disposed on the rotatable tip member.

10. The medical device of claim 1, wherein the catheter shaft has a plurality of slots formed therein.

11. A catheter assembly, comprising:
a hub body having a proximal end and a distal end;
wherein the hub body includes a plurality of fins extending at least partially between the proximal end and distal end thereof;
a catheter shaft attached to the distal end of the hub body; and
wherein the fins are configured to transfer torque from the hub body to the catheter shaft.

12. The catheter assembly of claim 11, wherein the hub body includes a rotatable distal tip that is rotatable relative to the hub body.

13. The catheter assembly of claim 12, further comprising one or more fins disposed on the rotatable distal tip.

14. The catheter assembly of claim 12, wherein the hub body includes an O-ring connector at the distal end for securing the rotatable distal tip to the hub body.

15. The catheter assembly of claim 11, further comprising one or more connectors disposed at the proximal end of the hub body.

16. The catheter assembly of claim 11, further comprising one or more wings disposed on the hub body.

17. The catheter assembly of claim 16, wherein the fins are disposed proximal to the wings.

18. The catheter assembly of claim 11, wherein the hub body includes at least eight fins.

19. The catheter assembly of claim 11, wherein the catheter shaft has a plurality of slots formed therein.

20. A method for rotating a catheter shaft relative to a hub, the method comprising:
providing a catheter assembly including a hub attached to the catheter shaft, the hub having a hub body including a proximal end, a distal end, and a lumen extending therebetween, wherein the hub includes a rotatable tip member, the rotatable tip member being rotatable relative to the hub body;
advancing the catheter shaft through a body lumen to a position adjacent to an area of interest; and
rotating the rotatable tip member, wherein rotating the rotatable tip member transfers torque from the rotatable tip member to the catheter shaft and rotates the catheter shaft.
